(19) United States
(12) Patent Application Publication
(10) Pub. No.: US 2018/0082104 A1
(43) Pub. Date: Mar. 22, 2018

(54) CLASSIFICATION OF CELLULAR IMAGES AND VIDEOS

(71) Applicant: Siemens Aktiengesellschaft, Munich (DE)

(72) Inventors: Shaohua Wan, Beijing (CN); Shanhui Sun, Princeton, NJ (US); Stefan Kluckner, Berlin (DE); Terrence Chen, Princeton, NJ (US); Ali Kamen, Skillman, NJ (US)

(21) Appl. No.: 15/554,295

(22) PCT Filed: Mar. 30, 2015
(86) PCT No.: PCT/US2015/023231

§ 371 Date: Aug. 29, 2017

Related U.S. Application Data
(60) Provisional application No. 62/126,823, filed on Mar. 2, 2015.

(51) Int. Cl. G06K 9/00; G06K 9/62; A61B 1/04; A61B 1/00; A61B 90/20

(52) U.S. Cl. CPC G06K 9/00147; G06K 9/0014; G06K 9/6223; G02B 21/008; A61B 1/04; A61B 1/00009; A61B 90/20; G06K 9/6276

(57) ABSTRACT

A method for performing cellular classification includes extracting a plurality of local feature descriptors from a set of input images and applying a coding process to covert each of the plurality of local feature descriptors into a multi-dimensional code. A feature pooling operation is applied on each of the plurality of local feature descriptors to yield a plurality of image representations and each image representation is classified as one of a plurality of cell types.

### CROSS-REFERENCE TO RELATED APPLICATIONS

This application claims priority to U.S. provisional application Ser. No. 62/126,823 filed Mar. 2, 2015, which is incorporated herein by reference in its entirety.

### TECHNICAL FIELD

The present disclosure relates generally to methods, systems, and apparatuses for performing for the classification of cellular images and videos. The proposed technology may be applied, for example, to classify endomicroscopy images and Digital Holographic Microscopy images.

### BACKGROUND

In-vivo cell imaging is the study of living cells using images acquired from imaging systems such as endomicroscopes. Due to recent advances in fluorescent protein and synthetic fluorophore technology, an increasing amount of research efforts are being devoted to in-vivo cell imaging techniques that provide insight into the fundamental nature of cellular and tissue function. In-vivo cell imaging technologies now span multiple modalities, including, for example, multi-photon, spinning disk microscopy, fluorescence, phase contrast, and differential interference contrast, and laser scanning confocal-based devices.

With the ever increasing amount of microscopy imaging data that is stored and processed digitally, one challenge is to categorize these images and make sense out of them reliably during medical procedures. Results obtained by these techniques may be used to support clinicians' manual/subjective analysis, leading to test results being more reliable and consistent. In conventional systems, results often must be acquired with a manual test procedure that is time-consuming and computing intensive. To this end, in order to address the shortcomings of the manual test procedure, it is desired to provide automated techniques (and related systems) to determine patterns in in-vivo cell images.

### SUMMARY

Embodiments of the present invention address and overcome one or more of the above shortcomings and drawbacks, by providing methods, systems, and apparatuses related to a feature coding process referred to herein as “Locality-Constrained Sparse Coding” (LSC). As described in further detail below, LSC not only enforces code sparsity for better discriminative power compared to conventional techniques, but LSC also preserves code locality in the sense that each descriptor is best coded within its local-coordinate system. These techniques may be applied to any coding-based image classification problem, including various cellular image and video classification problems.

According to some embodiments, a method for performing cellular classification includes extracting local feature descriptors from a set of input images and applying a coding process to covert each of the local feature descriptors into a multi-dimensional code. A feature pooling operation is applied on each of the plurality of local feature descriptors to yield image representations. Each image representation is then classified as one of a plurality of cell types. In some embodiments, the set of input images comprises a video stream whereby each image representation is classified using majority voting within a time window having a predetermined length.

Various techniques may be used for acquiring the set of input images. For example, in some embodiments, a plurality of input images is acquired, for example, via an endomicroscopy device or a digital holographic microscopy device during a medical procedure such as a complete blood count hematology examination. An entropy value is calculated for each of the plurality of input images. Each entropy value is representative of an amount of texture information in a respective image. Next, one or more low-entropy images are identified in the set of input images. These low-entropy images are each associated with a respective entropy value below a threshold value. Then, the set of input images is generated based on the plurality of input images, excluding the low-entropy images.

In some embodiments, the coding process used in the aforementioned method may use a generated codebook. For example, in some embodiments training features are extracted from a training set of images. A k-means clustering process is performed using the training features to yield feature clusters which are then used to generate a codebook. The exact implementation of the k-means clustering process may vary according to different embodiments. For example, in one embodiment, the k-means clustering process uses a Euclidean distance based on exhaustive nearest neighbor search to obtain the feature clusters. In other embodiments, the k-means clustering process uses a Euclidean distance based on a hierarchical vocabulary tree search to obtain the feature clusters. Once the codebook is generated, the coding process may use it to covert each of the local feature descriptors into the multi-dimensional code.

Additionally, it should be noted that the implementation of the coding process itself used in the aforementioned method may vary in different embodiments. In some embodiments, a sparse coding process may be used. For example, in one embodiment, the coding process is a Locality-constrained Linear Coding (LLC) coding process. In another embodiment, the coding process is a LSC coding process. In other embodiments, the coding process is a Bag of Words (BoW) coding process.

According to other embodiments, a second method for performing cellular classification includes generating a codebook prior to a medical procedure based on training image. During the medical procedure, a cell classification process is performed. This process may include acquiring an input image, for example, using an endomicroscopy device. Feature descriptors associated with the input image are determined and a coding process is applied to convert the plurality of feature descriptors into a coded dataset. A feature pooling operation is applied on the coded dataset to yield an image representation and a trained classifier is used to identify a class label corresponding to that image representation. The identified class label may be presented, for example, on a display operably coupled to the endomicroscopy device that acquired the input image. This class label may provide information such as, for example, an indication of whether biological material in the input image is malignant or benign.

The implementation of the coding process in the aforementioned second method may vary according to different embodiments. For example, in one embodiment, the coding process includes iteratively solving an optimization problem for each feature descriptor. This optimization problem may be configured to enforce code sparsity and code locality with respect to a respective feature descriptor. For example, in some embodiments, a k-nearest neighbor process is applied to each respective feature descriptor to identify a plurality of local bases. The code locality in each optimization problem may then be enforced using these local bases. Each optimization problem may be solved, for example, using a process such as Alternating Direction of Multipliers.

According to other embodiments, a system performing cellular classification includes a microscopy device, an imaging computer, and a display. The microscopy device is configured to acquire a set of input images during a medical procedure. Various types of microscopy devices known in the art may be used including, without limitation, a Confocal Laser Endo-microscopy device or a Digital Holographic Microscopy device. The imaging computer is configured to perform a cellular classification process during the medical procedure. This cellular classification process may include determining feature descriptors associated with the set of input images, applying a coding process to convert the feature descriptors into a coded dataset, applying a feature pooling operation on the coded dataset to yield an image representation, and using a trained classifier to identify a class label corresponding to the image representation. The display is configured to present the class label during the medical procedure.

Additional features and advantages of the invention will be made apparent from the following detailed description of illustrative embodiments that proceeds with reference to the accompanying drawings.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawings embodiments that are presently preferred, it being understood, however, that the invention is not limited to the specific instrumentalities disclosed. Included in the drawings are the following Figures:

FIG. 1 provides an example of a endomicroscopy-based system which may be used to perform cell classification, according to some embodiments;

FIG. 2 provides an overview of a Cell Classification Process that may be applied in some embodiments of the present invention;

FIG. 3 provides a set of low-entropy and high-entropy images of Glioblastoma and Meningioma;

FIG. 4 provides an example of image entropy distribution for images in a brain tumor dataset, as may be utilized in some embodiments;

FIG. 5 provides an example of an alternating projection method that may be used during filter learning, according to some embodiments;

FIG. 6 provides an example of cell images from a blood cell dataset that may be used in some embodiments;

FIG. 7A provides a table with detailed statistics of a white blood cell dataset for training and testing, as may be gathered using techniques described herein;

FIG. 7B provides a table with the recognition accuracy and speed of the different methods, when applied to the white blood cell dataset, according to some embodiments;

FIG. 8A provides an illustration of low-entropy and high-entropy images of Glioblastoma and Meningioma, as may be gathered and utilized in some embodiments;

FIG. 8B provides an illustration of recognition accuracy and speed of different classification methods on a brain tumor dataset, according to some embodiments;

FIG. 9 shows a graph illustrating the performance of majority voting-based classification with respect to time window size, according to some embodiments; and

FIG. 10 illustrates an exemplary computing environment, within which embodiments of the invention may be implemented.

### DETAILED DESCRIPTION OF ILLUSTRATIVE EMBODIMENTS

The following disclosure several embodiments directed at methods, systems, and apparatuses related to a feature coding process, referred to herein as Locality-constrained Sparse Coding (LSC) which utilizes a three part classification pipeline. These three parts include off-line unsupervised codebook learning, off-line, supervised classifier training and online image and video classification. Additionally, in some embodiments, a fast approximate solution to the LSC problem is determined based on k-nearest-neighbor (K-NN) search and Alternating Direction Method of Multiplier (ADMM). The various systems, methods, and apparatuses for cellular classification are described with reference to two cellular imaging modalities: Confocal Laser Endo-microscopy (CLE) and Digital Holographic Microscopy (DHM). However, it should be understood that the various embodiments of this disclosure are not limited to these modalities and may be applied in a variety of clinical settings. Additionally, it should be understood that the techniques described herein may be applied to the classification of various types of medical images, or even natural images.

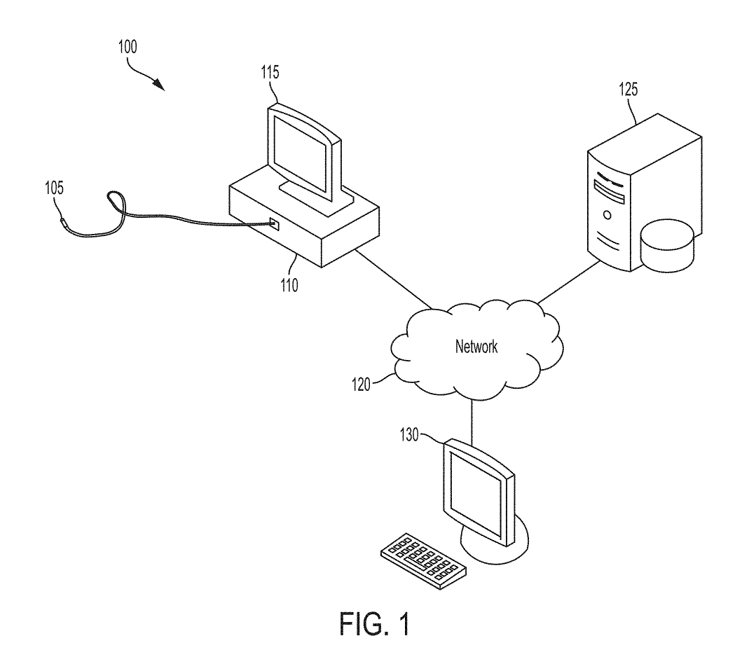

FIG. 1 provides an example of an endomicroscopy-based system 100 which may be used to perform feature coding with LSC, according to some embodiments. Briefly, endomicroscopy is a technique for obtaining histology-like images from inside the human body in real-time through a process known as “optical biopsy.” The term “endomicroscopy” generally refers to fluorescence confocal microscopy, although multi-photon microscopy and optical coherence tomography have also been adapted for endoscopic use and may be likewise used in various embodiments. Non-limiting examples of commercially available clinical endomicroscopes include the Pentax ISC-1000/EC3870CIK and Cellvizio (Mauna Kea Technologies, Paris, France). The main applications have traditionally been in imaging the gastro-intestinal tract, particularly for the diagnosis and characterization of Barrett's Esophagus, pancreatic cysts and colorectal lesions. The diagnostic spectrum of confocal endomicroscopy has recently expanded from screening and surveillance for colorectal cancer towards Barrett's esophagus, Helicobacter pylori associated gastritis and early gastric cancer. Endomicroscopy enables subsurface analysis of the gut mucosa and in-vivo histology during ongoing endoscopy in full resolution by point scanning laser fluorescence analysis. Cellular, vascular and connective structures can be seen in detail. The new detailed images seen with confocal laser endomicroscopy will allow a unique look on cellular structures and functions at and below the surface of the gut. Additionally, as discussed in further detail below, endomicroscopy may also be applied to brain surgery where identification of malignant (Glioblastoma) and benign (Meningioma) tumors from normal tissues is clinically important.

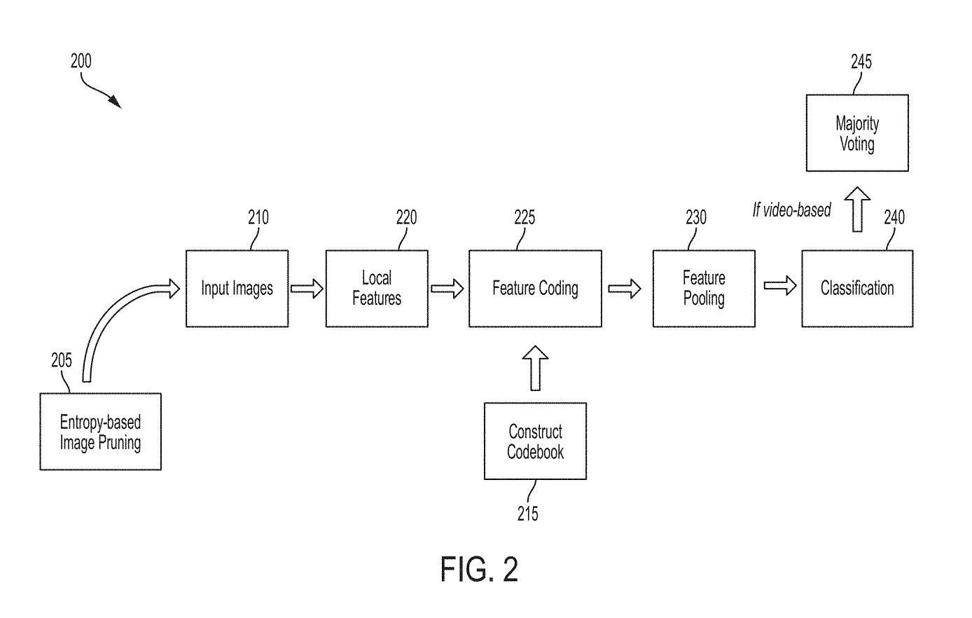

In the example of FIG. 1, a group of devices are configured to perform Confocal Laser Endo-microscopy (CLE). These devices include a Probe 105 operably coupled to an Imaging Computer 110 and an Imaging Display 115. In FIG. 1, Probe 105 is a confocal miniature probe. However, it should be noted that various types of miniature probes may be used, including probes designed for imaging various fields of view, imaging depths, distal tip diameters, and lateral and axial resolutions. The Imaging Computer 110 provides an excitation light or laser source used by the Probe 105 during imaging. Additionally, the Imaging Computer 110 may include imaging software to perform tasks such as recording, reconstructing, modifying, and/or export images gathered by the Probe 105. The Imaging Computer 110 may also be configured to perform a Cell Classification Process, discussed in greater detail below with respect to FIG. 2.

A foot pedal (not shown in FIG. 1) may also be connected to the Imaging Computer 110 to allow the user to perform functions such as, for example, adjusting the depth of confocal imaging penetration, start and stop image acquisition, and/or saving image either to a local hard drive or to a remote database such as Database Server 125. Alternatively or additionally, other input devices (e.g., computer, mouse, etc.) may be connected to the Imaging Computer 110 to perform these functions. The Imaging Display 115 receives images captured by the Probe 105 via the Imaging Computer 110 and presents those images for view in the clinical setting.

Continuing with the example of FIG. 1, the Imaging Computer 110 is connected (either directly or indirectly) to a Network 120. The Network 120 may comprise any computer network known in the art including, without limitation, an intranet or internet. Through the Network 120, the Imaging Computer 110 can store images, videos, or other related data on a remote Database Server 125. Additionally a User Computer 130 can communicate with the Imaging Computer 110 or the Database Server 125 to retrieve data (e.g., images, videos, or other related data) which can then be processed locally at the User Computer 130. For example, the User Computer 130 may retrieve data from either Imaging Computer 110 or the Database Server 125 and use it to perform the Cell Classification Process discussed below in FIG. 2.

Although FIG. 1 shows a CLE-based system, in other embodiments, the system may alternatively use a DHM imaging device. DHM, also known as interference phase microscopy, is an imaging technology that provides the ability to quantitatively track sub-nanometric optical thickness changes in transparent specimens. Unlike traditional digital microscopy, in which only intensity (amplitude) information about a specimen is captured, DHM captures both phase and intensity. The phase information, captured as a hologram, can be used to reconstruct extended morphological information (e.g., depth and surface characteristics) about the specimen using a computer algorithm. Modern DHM implementations offer several additional benefits, such as fast scanning/data acquisition speed, low noise, high resolution and the potential for label-free sample acquisition. While DHM was first described in the 1960s, instrument size, complexity of operation and cost has been major barriers to widespread adoption of this technology for clinical or point-of-care applications. Recent developments have attempted to address these barriers while enhancing key features, raising the possibility that DHM could be an attractive option as a core, multiple impact technology in healthcare and beyond.

The ability of DHM to achieve high-resolution, wide field imaging with extended depth and morphological information in a potentially label-free manner positions the technology for use in several clinical applications, including: hematology (e.g., RBC volume measurement, white blood cell differential, cell type classification), urine sediment analysis (e.g., scanning a microfluidic sample in layers to reconstruct the sediment and improving the classification accuracy of sediment constituents); tissue pathology (e.g., utilization of extended morphology/contrast of DHM to discriminate cancerous from healthy cells, in fresh tissue, without labeling); and rare cell detection (e.g., utilizing extended morphology/contrast of DHM to differentiate rare cells such as circulating tumor/epithelial cells, stem cells, infected cells, etc.). Given the latest advancements in DHM technology—particularly reductions in size, complexity and cost—these and other applications (including the Cell Classification Process described below in FIG. 2) can be performed within a clinical environment or at the point of care in a decentralized manner.

FIG. 2 provides an overview of a Cell Classification Process 200 which applies LSC, according to some embodiments of the present invention. This process 200 is illustrated as a pipeline of comprising three parts: off-line unsupervised codebook learning, off-line supervised classifier training, and online image and video classification. The core components of the process 200 are local feature extraction, feature coding, feature pooling and classification. Briefly, local feature points are detected on the input image, and descriptors are extracted from each feature point. These descriptors may include, for example, such as Local Binary Pattern (LBP), Scale Invariant Feature Transform (SIFT), Gabor features, and/or Histogram of Oriented Gradient (HOG). To encode local features, codebooks are learned offline. A codebook with m entries is applied to quantize each descriptor and generate the “code” layer. In some embodiments, K-means clustering method is utilized. For the supervised classification, each descriptor is then converted into an [symbol] ^{m} code. Finally, a classifier is trained using the coded features. This classifier may include any classifier known in the art including, for example, a support vector machine (SVM) and/or a random forest classifier. In some embodiments, where the input images are video-stream based, the process 100 is able to incorporate the visual cues from adjacent images. This significantly improves the performance of the process. In other embodiments, where the input images are low-contrast and contain little categorical information, the process is able to automatically discard those images from further processing. This increases the overall robustness of the process 100. Various components for performing the Cell Classification Process 200 are described in greater detail below, along with some additional optional features which may be applied in some embodiments.

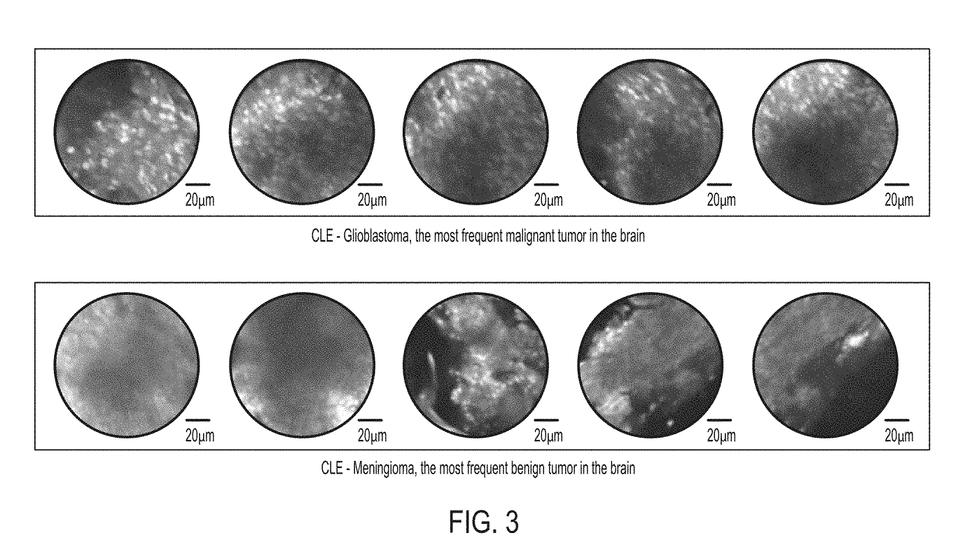

Prior to the start of the Cell Classification Process 200, a Entropy-based Image Pruning Component 205 may optionally be used to automatically remove image frames with low image texture information (e.g., low-contrast and contain little categorical information) that may not be clinically interesting or not suitable for image classification. This removal may be used, for example, to address the limited imaging capability of some CLE devices. Image entropy is a quantity which is used to describe the “informativeness” of an image, i.e., the amount of information contained in an image. Low-entropy images have very little contrast and large runs of pixels with the same or similar gray values. On the other hand, high entropy images have a great deal of contrast from one pixel to the next. FIG. 3 provides a set of low-entropy and high-entropy images of Glioblastoma and Meningioma. As shown in the figure, low-entropy images contain a lot of homogeneous image regions, while high-entropy images are characterized by rich image structures.

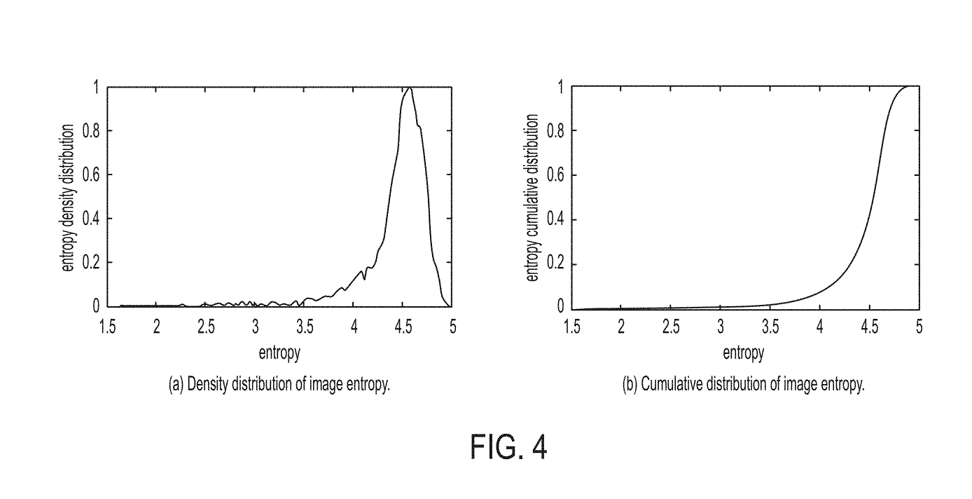

In some embodiments, the Entropy-based Image Pruning Component 205 performs pruning using an entropy threshold. This threshold may be set based on the distribution of the image entropy throughout the dataset. FIG. 4 provides an example of image entropy distribution for images in a brain tumor dataset, as may be utilized in some embodiments. As can be seen, there is a relatively large number of images whose entropy is significantly lower than that of the rest of the images. Thus, for this example, the entropy threshold can be set such that 10% images will be discarded from later stages of our system (e.g., 4.05 for data shown in FIG. 4).

Local Features 220 are extracted from one or more Input Images 210. Various techniques may be applied for feature extraction. In some embodiments, the Local Features 220 are extracted using human-designed features such as, without limitation, Scale Invariant Feature Transform (SIFT), Local Binary Pattern (LBP), Histogram of Oriented Gradient (HOG), and Gabor features. Each technique may be configured based on the clinical application and other user-desired characteristics of the results. For example, SIFT, a local feature descriptor that has been used for a large number of purposes in computer vision. It is invariant to translations, rotations and scaling transformations in the image domain and robust to moderate perspective transformations and illumination variations. Experimentally, the SIFT descriptor has been proven very useful in practice for image matching and object recognition under real-world conditions. In one embodiment, dense SIFT descriptors of 20×20 pixel patches computed over a grid with spacing of 10 pixels are utilized. Such dense image descriptors may be used to capture uniform regions in cellular structures such as low-contrast regions in case of Meningioma.

In some embodiments, rather than using human-designed features, machine learning techniques are used to automatically extract Local Features 220 based on filters that are learned from training images. These machine-learning techniques may use various detection techniques including, without limitation, edge detection, corner detection, blob detection, ridge detection, edge direction, change in intensity, motion detection, and shape detection.

Continuing with reference to FIG. 2, a Feature Coding Component 225 applies a coding process to convert each Local Feature 220 into an m-dimensional code cᵢ=[cᵢ₁ . . . , cᵢₘ]ε [symbol] ^{m} This conversion is performed using a codebook of m entries, B=[b₁ . . . , bₙ]ε [symbol] ^{d×n} generated offline by a Construct Codebook Component 215. Various techniques may be used for generating the codebook. For example, in some embodiments, k-means clustering performed on a random subset of large numbers (e.g. 100,000) of local features, extracted from a training set to form a visual vocabulary. Each feature cluster may be obtained, for example, by utilizing a Euclidean distance based exhaustive nearest-neighbor search or a hierarchical vocabulary tree structure (binary search tree).

Various types of coding processes may be employed by Feature Coding Component 225. Four example coding processes are described herein: Bag of Words (BoW), Sparse Coding, Locality-constrained Linear Coding (LLC), and Locality-constrained Sparse Coding (LSC). In some embodiments, the coding process employed by the Feature Coding Component 225 may help determine some of the parameters of the codebook generated by the Construct Codebook Component 215. For example, for a BoW scheme, the vocabulary tree structure with tree depth of 8 may be used. For Sparse Coding, LLC, and LSC, a k-means of Euclidean distance based exhaustive nearest neighbor search may be used.

Let X be a set of d-dimensional local descriptors extracted from an image (i.e., X=[x₁ . . . , xₙ]ε [symbol] ^{d×n}). Where BoW is employed as the coding process, for a local feature xᵢ, there is one and only one non-zero coding coefficient. The non-zero coding coefficient corresponds to the nearest visual word subject to a predefined distance. When the Euclidean distance is adopted, the code cᵢ may be calculated as:

$${c}_{ij} = \{ \begin{matrix}1 & if j = arg {min}_{j = 1 , … , n} {‖ {x}_{i} - {b}_{i} ‖}_{2}^{2} \\ 0 & otherwise\end{matrix}$$

In the Sparse Coding scheme, each local feature xᵢ is represented by a linear combination of a sparse set of basis vectors in the codebook. The coefficient vector cᵢ is obtained by solving an l₁-norm regularized problem:

$${c}_{i} = arg min {‖ {x}_{i} - {Bc}_{i} ‖}_{2}^{2} + λ {‖ {c}_{i} ‖}_{1}$$

$$s . t . {1}^{T} {c}_{i} = 1 , ∀ i$$

where ∥•∥ denotes the l₁-norm of the vector. The constraint 1^{T}cᵢ=1 follows the requirements of the sparse code.

Unlike Sparse Coding, LLC enforces codebook locality instead of sparsity. This leads to smaller coefficients for basis vectors farther away from xᵢ. The code cᵢ is computed by solving the following regularized least squares error:

$${c}_{i} = arg min {‖ {x}_{i} - {Bc}_{i} ‖}_{2}^{2} + λ {‖ {d}_{i} ⊙ {c}_{i} ‖}_{2}^{2}$$

$$s . t . {1}^{T} {c}_{i} = 1 , ∀ i$$

where ⊙ denotes the element-wise multiplication and dᵢε [symbol] ^{m} is the locality adaptor that gives different freedom for each basis vector proportional to its similarity to the input descriptor xᵢ. Specifically,

$${d}_{i} = exp \frac{dist ( {x}_{i} , B )}{σ} \qquad ( 4 )$$

where dis(xᵢ, B)=[dis(xᵢ, b₁) . . . dis(xᵢ, bₘ)]^{T}, and dis(xᵢ, bⱼ) is the Euclidean distance between xᵢ and bⱼ. The value of σ is used for adjusting the weight decay speed for local adaptation.

The LSC feature coding method compares favorably to conventional methods in that it not only enforces code sparsity for better discriminative power, but also preserves code locality in the sense that each descriptor is best coded within its local-coordinate system. Specifically, the LSC code can be formulated as:

$${c}_{i} = arg min {‖ {x}_{i} - {Bc}_{i} ‖}_{2}^{2} + λ {‖ {d}_{i} ⊙ {c}_{i} ‖}_{1} \\ s . t . {1}^{T} {c}_{i} = 1 , ∀ i \qquad ( 5 )$$

Although various algorithms exist for solving the conventional sparse coding problem, it becomes a significantly challenging optimization problem due to the locality weight vector dᵢ. In some embodiments, the Alternating Direction Method of Multipliers (ADMM) method is used to solve Equation 5. First, a dummy variable yᵢε [symbol] ^{m} is introduced so that Equation 5 may be reformulated as:

$${min}_{{c}_{i}} {‖ {x}_{i} - {Bc}_{i} ‖}_{2}^{2} + λ {‖ {d}_{i} ⊙ {y}_{i} ‖}_{1} \\ s . t . {1}^{T} {y}_{i} = 1 \\ {c}_{i} = {y}_{i} \qquad ( 6 )$$

Then, we can form the augmented Lagrangian of the above objective, which becomes

$${min}_{{c}_{i} , {y}_{i}} ℒ ( {c}_{i} , {y}_{i} ) = {‖ {x}_{i} - {By}_{i} ‖}_{2}^{2} + λ {‖ {d}_{i} ⊙ {c}_{i} ‖}_{1} + μ {‖ {c}_{i} - {y}_{i} ‖}_{2}^{2} + {ρ}^{T} ( {c}_{i} - {y}_{i} ) + μ {‖ {1}^{T} {y}_{i} - 1 ‖}_{2}^{2} + γ ( {1}^{T} {y}_{i} - 1 ) \qquad ( 7 )$$

The ADMM includes three iterations:

$$\begin{matrix}\text{?} = arg {min}_{{y}_{i}} ℒ ( \text{?} ) & ( 8 a ) \\ \text{?} = arg {min}_{{c}_{i}} ℒ ( {y}_{i}^{t + 1} , {c}_{i} , {ρ}^{t} , {γ}^{t} ) & ( 8 b ) \\ {ρ}^{t + 1} = {ρ}^{t} + μ ( {c}_{i} - {y}_{i} ) , {γ}^{t + 1} = {γ}^{t} + μ ( {1}^{T} {y}_{i} - 1 ) \\ \text{?} \text{indicates text missing or illegible when filed} & ( 8 c )\end{matrix}$$

which allows the original problem to be broken into a sequence of sub-problems. In sub-problem 8a, we are minimizing [symbol] (yᵢ,cᵢ^{t},ρ^{t},γ^{t}) w.r.t. only yᵢ• and the l₁-penalty ∥dᵢ⊙cᵢ∥₁ disappears from the objective making it a very efficient and simple least-squares regression problem. In sub-problem 8b, we are minimizing [symbol] (yᵢ^{t+1},cᵢ,ρ^{t},γ^{t}) w.r.t. only cᵢ, and the term ∥xᵢ−Byᵢ∥₂²+μ∥1^{T}yᵢ−1∥₂²+γ(1^{T}yᵢ−1) disappears allowing cᵢ to be solved independently across each element. This now allows soft-thresholding to be used more efficiently. The current estimates of yᵢ and cᵢ are then combined in sub-problem 8c to update the current estimate of the Lagrangian multipliers ρ and γ. Note that ρ and γ play a special role here, as they allow us to employ an imperfect estimate of ρ and γ when solving for both yᵢ and cᵢ. For convenience, the following soft-thresholding (shrinkage) operator: may be employed:

$$\text{?} = \{ \begin{matrix}\text{?} & \text{?} \\ x + \text{?} & \text{?} \\ 0 & otherwise\end{matrix} \\ \text{?} \text{indicates text missing or illegible when filed} \qquad ( 9 )$$

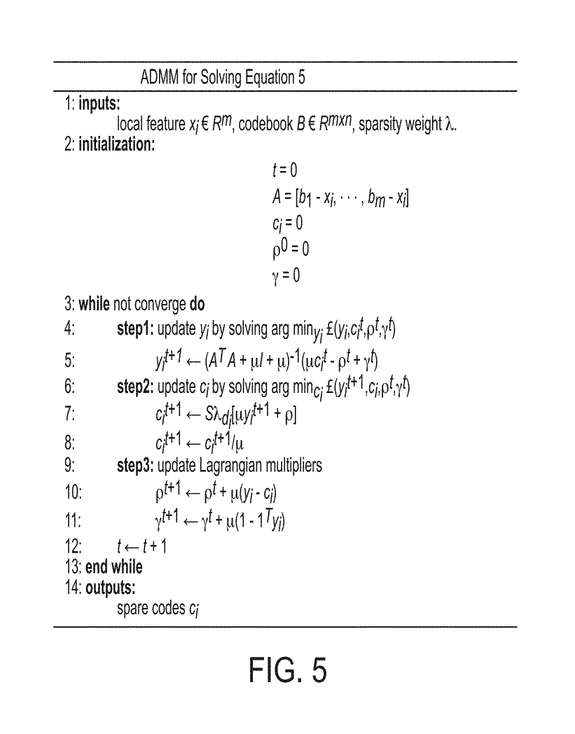

FIG. 5 provides additional detail of the algorithm for solving Equation 5, according to some embodiments. The size of the codebook B has a direct effect on the time complexity of the algorithm. To develop a fast approximate solution to LSC, we can simply use the K (K<n) nearest neighbors of xᵢ as the local bases Bᵢ, and solve a much smaller sparse reconstruction system to get the codes:

$${\hat{c}}_{i} = arg min {‖ {x}_{i} - {B}_{i} {\hat{c}}_{i} ‖}_{2}^{2} + λ {‖ {\hat{d}}_{i} ⊙ {\hat{c}}_{i} ‖}_{1} \\ s . t . {1}^{T} \hat{c} = 1 , ∀ t \qquad ( 10 )$$

As K is usually very small, solving Equation 10 is very fast. For searching K-nearest neighbors, one can apply a simple but efficient hierarchical K-NN search strategy. In this way, a much larger codebook can be used to improve the modelling capacity, while the computation in LSC remains fast and efficient.

Returning to FIG. 2, a Feature Pooling Component 230 applies one or more feature pooling operations to summarize the feature maps to generate the final image representation. The Feature Pooling Component 230 may apply any pooling technique known in the art including, for example, max-pooling, average-pooling, or a combination thereof. For example, in some embodiments, the Feature Pooling Component 230 uses a composition of max-pooling and average-pooling operations. For example, each feature map may be partitioned into regularly spaced square patches and a max-polling operation may be applied (i.e., the maximum response for the feature over each square patch may be determined). The max-pooling operation allows local invariance to translation. Then, the average of the maximum response may be calculated from the square patches, i.e. average pooling is applied after max-pooling. Finally, the image representation may be formed by aggregating feature responses from the average-pooling operation.

The Classification Component 240 identifies one or more class labels for the final image representation based on one or more pre-defined criteria. The Classification Component 240 utilizes one or more classifier algorithms which may be trained and configured based on the clinical study. For example, in some embodiments, the classifier is trained using a brain tumor dataset, such that it can label images as either Glioblastoma or Meningioma. Various types of classifier algorithms may be used by the Classification Component 240 including, without limitation, support vector machines (SVM), k-nearest neighbors (k-NN), and random forests. Additionally, different types of classifiers can be used in combination.

For video image sequences, a Majority Voting Component 245 may optionally perform a majority voting based classification scheme that boosts the recognition performance for the video stream. Thus, if input images are video-stream based, the process 200 is able to incorporate the visual cues from adjacent images. The Majority Voting Component 245 assigns class labels to the current image using the majority voting result of the images within a fixed length time window surrounding the current frame in a causal fashion. The length of the window may be configured based on user input. For example, the user may provide a specific length value or clinical setting which may be used to derive such a value. Alternatively, the length may be dynamically adjusted over time based on an analysis of past results. For example, if the user indicates that the Majority Voting Component 245 is providing inadequate or sub-optimal results, the window maybe adjusted by modifying the window size by a small value. Over time, the Majority Voting Component 245 can learn an optimal window length for each type of data being processed by the Cell Classification Process 200.

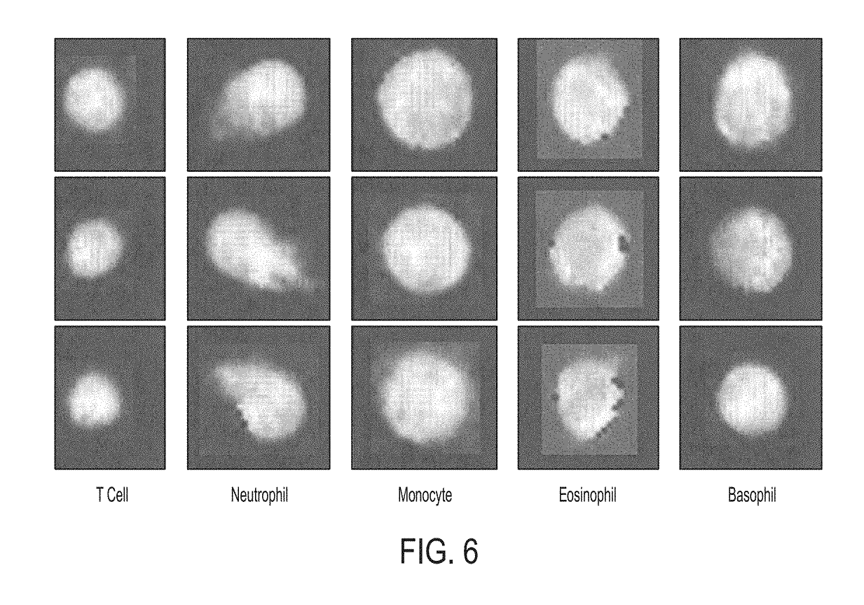

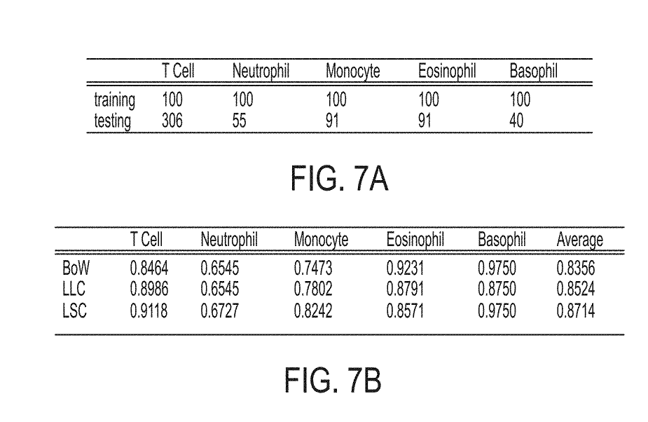

As an example application of the Cell Classification Process 200, consider a White Blood Cell dataset which comprises images of five white blood cell categories, including T-Cell, Neutrophil, Monocyte, Eosinophil, and Basophil. An example of such a dataset is provided in FIG. 6. The image size is 120×120. Experiments were performed to evaluate the differences of using BoW, LLC, and LSC, respectively with the Cell Classification Process. FIG. 7A provides a table with detailed statistics of the Blood Cell dataset for training and testing. FIG. 7B provides a table with the recognition accuracy and speed of the different methods, when applied to the White Blood Cell dataset. As shown in FIG. 7B, LSC provides recognition which is as good, if not better, than BoW and LLC for almost all of the cases.

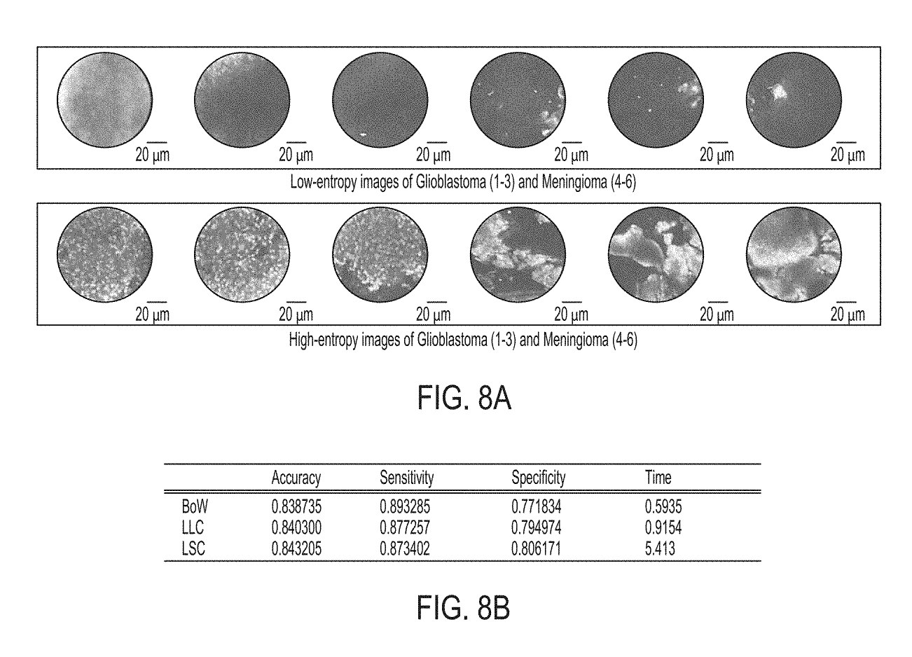

As another example, consider endomicroscopic videos collected using a CLE Device (see FIG. 1) that is inserted inside the patients' brain for examining brain tumor tissues. This collection may result in a set of videos for Glioblastoma and a set of videos for Meningioma. One example of the images collected in such videos is provided in FIG. 3. To evaluate the performance of the techniques discussed herein, an analysis was performed using the leave-one-video-out approach. More specifically, as a first step, 10 Glioblastoma and 10 Meningioma sequences were randomly selected. Next, as a second step, one pair of sequences from that first set were selected for testing and the remaining sequences for training. Then, as a third step, 4000 Glioblastoma frames and 4000 Meningioma frames are selected from the training sets. The experiment was repeated 10 times. Since brain tumors are visible only within the circle region of the microscope, a circle mask is applied to each image and local features are only extracted from within the circle mask, as shown in FIG. 8A. FIG. 8B shows a table detailing the recognition accuracy and speed of different techniques described herein when applied to the brain tumor dataset.

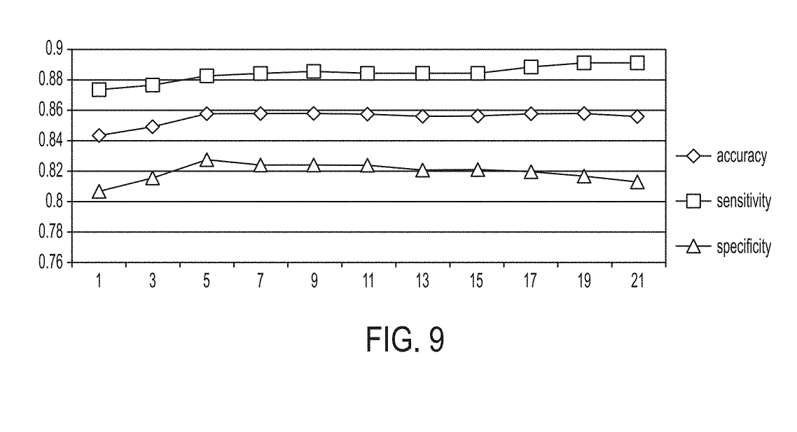

Additionally, the technique for majority voting described herein may also be illustrated with the brain tumor dataset. FIG. 9 shows a graph illustrating the performance of majority voting-based classification with respect to time window size. In this example, the sliding time window is set to T in length and the class label for the current frame is derived using the majority voting result of the frames within the sliding time window. The recognition performance with respect to the time window length T is given in chart illustrated in FIG. 9. In this example, the optimal performance is achieved at T=5. It is quite likely that higher recognition accuracy can be achieved using much longer time window. In practice, however, one has to balance the relative importance between recognition, speed and accuracy.

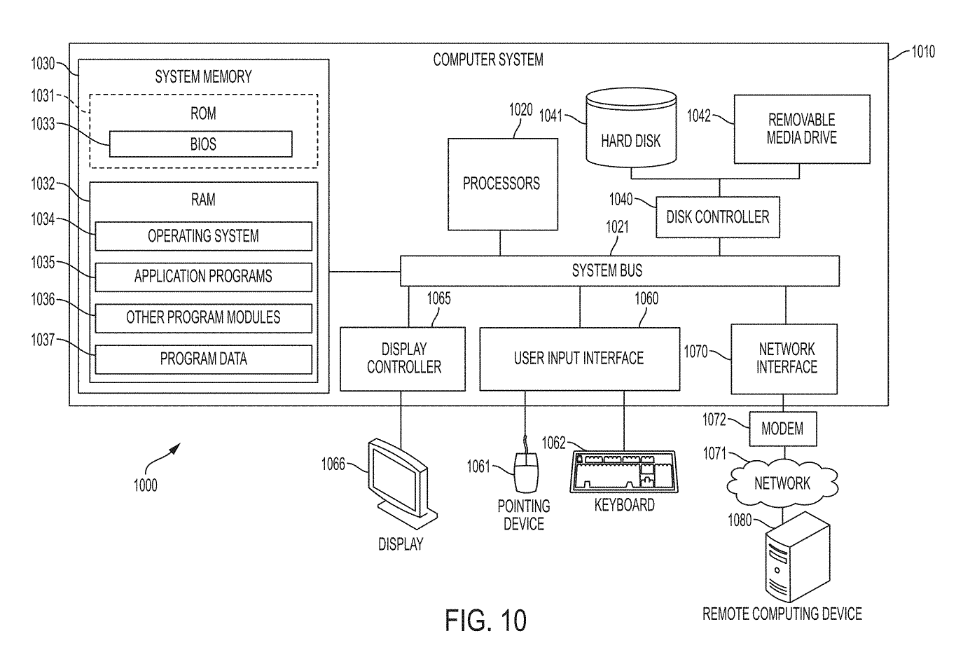

FIG. 10 illustrates an exemplary computing environment 1000 within which embodiments of the invention may be implemented. For example, this computing environment 1000 may be used to implement one or more devices shown in FIG. 1 and execute the Cell Classification Process 200 described in FIG. 2. The computing environment 1000 may include computer system 1010, which is one example of a computing system upon which embodiments of the invention may be implemented. Computers and computing environments, such as computer system 1010 and computing environment 1000, are known to those of skill in the art and thus are described briefly here.

As shown in FIG. 10, the computer system 1010 may include a communication mechanism such as a bus 1021 or other communication mechanism for communicating information within the computer system 1010. The computer system 1010 further includes one or more processors 1020 coupled with the bus 1021 for processing the information. The processors 1020 may include one or more central processing units (CPUs), graphical processing units (GPUs), or any other processor known in the art.

The computer system 1010 also includes a system memory 1030 coupled to the bus 1021 for storing information and instructions to be executed by processors 1020. The system memory 1030 may include computer readable storage media in the form of volatile and/or nonvolatile memory, such as read only memory (ROM) 1031 and/or random access memory (RAM) 1032. The system memory RAM 1032 may include other dynamic storage device(s) (e.g., dynamic RAM, static RAM, and synchronous DRAM). The system memory ROM 1031 may include other static storage device(s) (e.g., programmable ROM, erasable PROM, and electrically erasable PROM). In addition, the system memory 1030 may be used for storing temporary variables or other intermediate information during the execution of instructions by the processors 1020. A basic input/output system 1033 (BIOS) containing the basic routines that help to transfer information between elements within computer system 1010, such as during start-up, may be stored in ROM 1031. RAM 1032 may contain data and/or program modules that are immediately accessible to and/or presently being operated on by the processors 1020. System memory 1030 may additionally include, for example, operating system 1034, application programs 1035, other program modules 1036 and program data 1037.

The computer system 1010 also includes a disk controller 1040 coupled to the bus 1021 to control one or more storage devices for storing information and instructions, such as a hard disk 1041 and a removable media drive 1042 (e.g., floppy disk drive, compact disc drive, tape drive, and/or solid state drive). The storage devices may be added to the computer system 1010 using an appropriate device interface (e.g., a small computer system interface (SCSI), integrated device electronics (IDE), Universal Serial Bus (USB), or FireWire).

The computer system 1010 may also include a display controller 1065 coupled to the bus 1021 to control a display 1066, such as a cathode ray tube (CRT) or liquid crystal display (LCD), for displaying information to a computer user. The computer system includes an input interface 1060 and one or more input devices, such as a keyboard 1062 and a pointing device 1061, for interacting with a computer user and providing information to the processor 1020. The pointing device 1061, for example, may be a mouse, a trackball, or a pointing stick for communicating direction information and command selections to the processor 1020 and for controlling cursor movement on the display 1066. The display 1066 may provide a touch screen interface which allows input to supplement or replace the communication of direction information and command selections by the pointing device 1061.

The computer system 1010 may perform a portion or all of the processing steps of embodiments of the invention in response to the processors 1020 executing one or more sequences of one or more instructions contained in a memory, such as the system memory 1030. Such instructions may be read into the system memory 1030 from another computer readable medium, such as a hard disk 1041 or a removable media drive 1042. The hard disk 1041 may contain one or more datastores and data files used by embodiments of the present invention. Datastore contents and data files may be encrypted to improve security. The processors 1020 may also be employed in a multi-processing arrangement to execute the one or more sequences of instructions contained in system memory 1030. In alternative embodiments, hard-wired circuitry may be used in place of or in combination with software instructions. Thus, embodiments are not limited to any specific combination of hardware circuitry and software.

As stated above, the computer system 1010 may include at least one computer readable medium or memory for holding instructions programmed according to embodiments of the invention and for containing data structures, tables, records, or other data described herein. The term “computer readable medium” as used herein refers to any medium that participates in providing instructions to the processor 1020 for execution. A computer readable medium may take many forms including, but not limited to, non-volatile media, volatile media, and transmission media. Non-limiting examples of non-volatile media include optical disks, solid state drives, magnetic disks, and magneto-optical disks, such as hard disk 1041 or removable media drive 1042. Non-limiting examples of volatile media include dynamic memory, such as system memory 1030. Non-limiting examples of transmission media include coaxial cables, copper wire, and fiber optics, including the wires that make up the bus 1021. Transmission media may also take the form of acoustic or light waves, such as those generated during radio wave and infrared data communications.

The computing environment 1000 may further include the computer system 1010 operating in a networked environment using logical connections to one or more remote computers, such as remote computer 1080. Remote computer 1080 may be a personal computer (laptop or desktop), a mobile device, a server, a router, a network PC, a peer device or other common network node, and typically includes many or all of the elements described above relative to computer system 1010. When used in a networking environment, computer system 1010 may include modem 1072 for establishing communications over a network 1071, such as the Internet. Modem 1072 may be connected to bus 1021 via user network interface 1070, or via another appropriate mechanism.

Network 1071 may be any network or system generally known in the art, including the Internet, an intranet, a local area network (LAN), a wide area network (WAN), a metropolitan area network (MAN), a direct connection or series of connections, a cellular telephone network, or any other network or medium capable of facilitating communication between computer system 1010 and other computers (e.g., remote computer 1080). The network 1071 may be wired, wireless or a combination thereof. Wired connections may be implemented using Ethernet, Universal Serial Bus (USB), RJ-11 or any other wired connection generally known in the art. Wireless connections may be implemented using Wi-Fi, WiMAX, and Bluetooth, infrared, cellular networks, satellite or any other wireless connection methodology generally known in the art. Additionally, several networks may work alone or in communication with each other to facilitate communication in the network 1071.

The embodiments of the present disclosure may be implemented with any combination of hardware and software. In addition, the embodiments of the present disclosure may be included in an article of manufacture (e.g., one or more computer program products) having, for example, computer-readable, non-transitory media. The media has embodied therein, for instance, computer readable program code for providing and facilitating the mechanisms of the embodiments of the present disclosure. The article of manufacture can be included as part of a computer system or sold separately.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

An executable application, as used herein, comprises code or machine readable instructions for conditioning the processor to implement predetermined functions, such as those of an operating system, a context data acquisition system or other information processing system, for example, in response to user command or input. An executable procedure is a segment of code or machine readable instruction, sub-routine, or other distinct section of code or portion of an executable application for performing one or more particular processes. These processes may include receiving input data and/or parameters, performing operations on received input data and/or performing functions in response to received input parameters, and providing resulting output data and/or parameters.

A graphical user interface (GUI), as used herein, comprises one or more display images, generated by a display processor and enabling user interaction with a processor or other device and associated data acquisition and processing functions. The GUI also includes an executable procedure or executable application. The executable procedure or executable application conditions the display processor to generate signals representing the GUI display images. These signals are supplied to a display device which displays the image for viewing by the user. The processor, under control of an executable procedure or executable application, manipulates the GUI display images in response to signals received from the input devices. In this way, the user may interact with the display image using the input devices, enabling user interaction with the processor or other device.

The functions and process steps herein may be performed automatically or wholly or partially in response to user command. An activity (including a step) performed automatically is performed in response to one or more executable instructions or device operation without user direct initiation of the activity.

The system and processes of the figures are not exclusive. Other systems, processes and menus may be derived in accordance with the principles of the invention to accomplish the same objectives. Although this invention has been described with reference to particular embodiments, it is to be understood that the embodiments and variations shown and described herein are for illustration purposes only. Modifications to the current design may be implemented by those skilled in the art, without departing from the scope of the invention. As described herein, the various systems, subsystems, agents, managers and processes can be implemented using hardware components, software components, and/or combinations thereof. No claim element herein is to be construed under the provisions of 35 U.S.C. 112, sixth paragraph, unless the element is expressly recited using the phrase “means for.”

We claim:

1. A method for performing cellular classification, the method comprising:
extracting a plurality of local feature descriptors from a set of input images;
converting each local feature descriptor into a multi
applying a Locality-constrained Sparse Coding (LSC) coding process to covert each of the plurality of local feature descriptors into a multi-dimensional code using a codebook, wherein the LSC coding process iteratively solves an optimization problem which enforces code sparsity and code locality with respect to each local feature descriptor and the codebook;
applying a feature pooling operation on each of the plurality of local feature descriptors to yield a plurality of image representations; and
classifying each image representation as one of a plurality of cell types.

2. The method of claim 1, further comprising:
acquiring a plurality of input images;
calculating an entropy value for each of the plurality of input images, each entropy value representative of an amount of texture information in a respective image;
identifying one or more low-entropy images in the set of input images, wherein the one or more low-entropy images are each associated with a respective entropy value below a threshold value; and
generating the set of input images based on the plurality of input images, wherein the set of input images excludes the one or more low-entropy images.

3. The method of claim 2, wherein the plurality of input images are acquired using an endomicroscopy device during a medical procedure.

4. The method of claim 2, wherein the plurality of input images are acquired using a digital holographic microscopy device during a complete blood count hematology examination.

5. The method of claim 1, further comprising:
extracting a plurality of training features from a training set of images;
performing a k-means clustering process using the plurality of training features to yield a plurality of feature clusters; and
generating the codebook based on the plurality of feature clusters,
wherein the coding process uses the codebook to covert each of the plurality of local feature descriptors into the multi-dimensional code.

6. The method of claim 5, wherein the k-means clustering process uses a Euclidean distance based on exhaustive nearest neighbor search to obtain the plurality of feature clusters.

7. (canceled)

8. (canceled)

9. (canceled)

10. The method of claim 5, wherein the k-means clustering process uses a Euclidean distance based on a hierarchical vocabulary tree search to obtain the plurality of feature clusters.

11. (canceled)

12. The method of claim 1, wherein the set of input images comprises a video stream and each image representation is classified using majority voting within a time window having a predetermined length.

13. A method for performing cellular classification during a medical procedure, the method comprising:
prior to the medical procedure, generating a codebook based on a plurality of training images; and
during the medical procedure, performing a cell classification process comprising:
acquiring an input image using an endomicroscopy device,
determining a plurality of feature descriptors associated with the input image;
applying a Locality-constrained Sparse Coding (LSC) coding process to convert the plurality of feature descriptors into a coded dataset using the codebook, wherein the LSC coding process iteratively solves an optimization problem which enforces code sparsity and code locality with respect to each respective feature descriptor and the codebook;
applying a feature pooling operation on the coded dataset to yield an image representation,
using a trained classifier to identify a class label corresponding to the image representation, and
presenting the class label on a display operably coupled to the endomicroscopy device.

14. (canceled)

15. The method of claim 13, wherein the optimization problem is solved using an Alternating Direction of Multipliers process.

16. The method of claim 15, further comprising:
applying a k-nearest neighbor process to the respective feature descriptor to identify a plurality of local bases,
wherein the code locality in each optimization problem is enforced using the plurality of local bases.

17. The method of claim 13, wherein the class label provides an indication of whether biological material in the input image is malignant or benign.

18. A system performing cellular classification, the system comprising:
a microscopy device configured to acquire a set of input images during a medical procedure;
an imaging computer configured to perform a cellular classification process during the medical procedure, the cellular classification process comprising:
determining a plurality of feature descriptors associated with the set of input images,
applying a Locality-constrained Sparse Coding (LSC) coding process to convert the plurality of feature descriptors into a coded dataset using a codebook, wherein the LSC coding process iteratively solves an optimization problem which enforces code sparsity and code locality with respect to each respective feature descriptor and the codebook;
applying a feature pooling operation on the coded dataset to yield an image representation,
using a trained classifier to identify a class label corresponding to the image representation, and
a display configured to present the class label during the medical procedure.

19. The system of claim 18, wherein the microscopy device is a Confocal Laser Endo-microscopy device.

20. The system of claim 18, wherein the microscopy device is a Digital Holographic Microscopy device.
